(19) United States
(12) Patent Application Publication
(10) Pub. No.: US 2013/0075447 A1
(43) Pub. Date: Mar. 28, 2013

(54) ADJUNCT THERAPY DEVICE FOR APPLYING HEMOSTATIC AGENT

(71) Applicants: William B. Weisenburgh, II, Maineville, OH (US); Craig S. Smith, Cincinnati, OH (US); Gregory B. Blair, San Jose, CA (US); Zhifan F. Huang, Mason, OH (US); Douglas B. Hoffman, Harrison, OH (US); Kristi S. Geier, Morrow, OH (US); Bret W. Smith, Kings Mills, OH (US); Thomas W. Lytle, IV, Liberty Township, OH (US); Mark D. Overmyer, Cincinnati, OH (US); Brian W. Bear, Cincinnati, OH (US); Michael E. Setser, Burlington, KY (US); Thu Anh Le, Bridgewater, NJ (US); James A. Woodard, JR., Mason, OH (US); Kreena R. Modi, Akron, OH (US); Joseph Zavatsky, Flemington, NJ (US)

(72) Inventors: William B. Weisenburgh, II, Maineville, OH (US); Craig S. Smith, Cincinnati, OH (US); Gregory B. Blair, San Jose, CA (US); Zhifan F. Huang, Mason, OH (US); Douglas B. Hoffman, Harrison, OH (US); Kristi S. Geier, Morrow, OH (US); Bret W. Smith, Kings Mills, OH (US); Thomas W. Lytle, IV, Liberty Township, OH (US); Mark D. Overmyer, Cincinnati, OH (US); Brian W. Bear, Cincinnati, OH (US); Michael E. Setser, Burlington, KY (US); Thu Anh Le, Bridgewater, NJ (US); James A. Woodard, JR., Mason, OH (US); Kreena R. Modi, Akron, OH (US); Joseph Zavatsky, Flemington, NJ (US)

(21) Appl. No.: 13/240,141

(22) Filed: Sep. 22, 2011

(51) Int. Cl. A61B 17/068

(52) U.S. Cl. 227/176.1

(57) ABSTRACT

A surgical instrument includes a handle portion, a shaft housing a firing bar, an end effector comprising an anvil, a lower jaw, and a stapling and severing assembly responsive to a longitudinal closing motion produced by the handle portion and the shaft. The lower jaw is configured to receive a removable cartridge. The cartridge includes a housing, a plurality of staples disposed in the housing, and a deck disposed over the plurality of staples. The deck defines apertures, with each aperture being substantially disposed over each staple. The cartridge further receives a buttress material stored in one or both of the anvil or cartridge. The material is releasable onto severed tissue via a firing bar severing the buttress material in response to the longitudinal closing motion.

### BACKGROUND

In some settings, endoscopic surgical instruments may be preferred over traditional open surgical devices since a smaller incision may reduce the post-operative recovery time and complications. Consequently, some endoscopic surgical instruments may be suitable for placement of a distal end effector at a desired surgical site through a cannula of a trocar. These distal end effectors may engage tissue in a number of ways to achieve a diagnostic or therapeutic effect (e.g., endocutter, grasper, cutter, stapler, clip applier, access device, drug/gene therapy delivery device, and energy delivery device using ultrasound, RF, laser, etc.). Endoscopic surgical instruments may include a shaft between the end effector and a handle portion, which is manipulated by the clinician. Such a shaft may enable insertion to a desired depth and rotation about the longitudinal axis of the shaft, thereby facilitating positioning of the end effector within the patient. Positioning of an end effector may be further facilitated through inclusion of one or more articulation joints or features, enabling the end effector to be selectively articulated or otherwise deflected relative to the longitudinal axis of the shaft.

Examples of endoscopic surgical instruments include surgical staplers. Some such staplers are operable to clamp down on layers of tissue, cut through the clamped layers of tissue, and drive staples through the layers of tissue to substantially seal the severed layers of tissue together near the severed ends of the tissue layers. Merely exemplary surgical staplers are disclosed in; U.S. Pat. No. 4,805,823, entitled “Pocket Configuration for Internal Organ Staplers,” issued Feb. 21, 1989; U.S. Pat. No. 5,415,334, entitled “Surgical Stapler and Staple Cartridge,” issued May 16, 1995; U.S. Pat. No. 5,465,895, entitled “Surgical Stapler Instrument,” issued Nov. 14, 1995; U.S. Pat. No. 5,597,107, entitled “Surgical Stapler Instrument,” issued Jan. 28, 1997; U.S. Pat. No. 5,632,432, entitled “Surgical Instrument,” issued May 27, 1997; U.S. Pat. No. 5,673,840, entitled “Surgical Instrument,” issued Oct. 7, 1997; U.S. Pat. No. 5,704,534, entitled “Articulation Assembly for Surgical Instruments,” issued Jan. 6, 1998; U.S. Pat. No. 5,814,055, entitled “Surgical Clamping Mechanism,” issued Sep. 29, 1998; U.S. Pat. No. 6,964,363, entitled “Surgical Stapling Instrument having Articulation Joint Support Plates for Supporting a Firing Bar,” issued Nov. 15, 2005; U.S. Pat. No. 6,978,921, entitled “Surgical Stapling Instrument Incorporating an E-Beam Firing Mechanism,” issued Dec. 27, 2005; U.S. Pat. No. 6,988,649, entitled “Surgical Stapling Instrument Having a Spent Cartridge Lockout,” issued Jan. 24, 2006; U.S. Pat. No. 7,000,818, entitled “Surgical Stapling Instrument Having Separate Distinct Closing and Firing Systems,” issued Feb. 21, 2006; U.S. Pat. No. 7,111,769, entitled “Surgical Instrument Incorporating an Articulation Mechanism having Rotation about the Longitudinal Axis,” issued Sep. 26, 2006; U.S. Pat. No. 7,143,923, entitled “Surgical Stapling Instrument Having a Firing Lockout for an Unclosed Anvil,” issued Dec. 5, 2006; U.S. Pat. No. 7,303,108, entitled “Surgical Stapling Instrument Incorporating a Multi-Stroke Firing Mechanism with a Flexible Rack,” issued Dec. 4, 2007; U.S. Pat. No. 7,367,485, entitled “Surgical Stapling Instrument Incorporating a Multistroke Firing Mechanism Having a Rotary Transmission,” issued May 6, 2008; U.S. Pat. No. 7,380,695, entitled “Surgical Stapling Instrument Having a Single Lockout Mechanism for Prevention of Firing,” issued Jun. 3, 2008; U.S. Pat. No. 7,380,696, entitled “Articulating Surgical Stapling Instrument Incorporating a Two-Piece E-Beam Firing Mechanism,” issued Jun. 3, 2008; U.S. Pat. No. 7,404,508, entitled “Surgical Stapling and Cutting Device,” issued Jul. 29, 2008; U.S. Pat. No. 7,434,715, entitled “Surgical Stapling Instrument having Multistroke Firing with Opening Lockout,” issued Oct. 14, 2008; U.S. Pat. No. 7,721,930, entitled “Disposable Cartridge with Adhesive for Use with a Stapling Device,” issued May 25, 2010; and U.S. Pat. No. 7,455,208, entitled “Surgical Instrument with Articulating Shaft with Rigid Firing Bar Supports,” issued Nov. 25, 2008. The disclosure of each of the above-cited U.S. patents is incorporated by reference herein. While the surgical staplers referred to above are described as being used in endoscopic procedures, it should be understood that such surgical staplers may also be used in open procedures and/or other non-endoscopic procedures.

While various kinds of surgical stapling instruments and associated components have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1A depicts a perspective view of an articulating surgical instrument with an end effector in a nonarticulated position;

FIG. 1B depicts a perspective view of the surgical instrument of FIG. 1A with an end effector in an articulated position;

FIG. 2 depicts a perspective view of an opened end effector of the surgical instrument of FIGS. 1A-1B;

FIG. 3A depicts a side cross-sectional view of the end effector of FIG. 2, taken along line 3-3 of FIG. 2, with the firing bar in a proximal position;

FIG. 3B depicts a side cross-sectional view of the end effector of FIG. 2, taken along line 3-3 of FIG. 2, but showing the firing bar in a distal position;

FIG. 4 depicts an end cross-sectional view of the end effector of FIG. 2, taken along line 4-4 of FIG. 2;

FIG. 5 depicts an exploded perspective view of the end effector of FIG. 2;

FIG. 6 depicts a perspective view of the end effector of FIG. 2, positioned at tissue and having been actuated once in the tissue;

FIG. 7 depicts a perspective view of a version of removable cartridge of the end effector of FIG. 2 with an exemplary buttress disposed above the cartridge;

FIG. 8 depicts a detail view of an exemplary staple from the end effector of FIG. 2 being actuated through tissue;

FIG. 9 depicts a perspective view of an alternative version of an anvil of an upper jaw and cartridge in a lower jaw of the end effector of FIG. 2 and an exemplary applicator to dispense a biocompatible material onto the anvil and lower jaw;

FIG. 10 depicts an elevation view of the end effector of FIG. 9 with the biocompatible material dispensed onto the anvil and lower jaw;

FIG. 11A depicts a cross-sectional end view of the dispenser of FIG. 9 taken along line 11-11 of FIG. 9;

FIG. 11B depicts a cross-sectional end view of an alternate version of the dispenser of FIG. 9 taken along line 11-11 of FIG. 9;

FIG. 12 depicts an elevation view of an exemplary staple released from the end effector of FIG. 10 into tissue;

FIG. 13 depicts a cross-sectional side view of an exemplary removable cartridge inserted into a lower jaw of the end effector of FIG. 2 inserted a staple through an exemplary buttress and into tissue;

FIG. 14 depicts a fragmentary, perspective view of the cartridge of FIG. 13;

FIG. 15 depicts a perspective view of an end effector with an anvil cartridge including a tissue repair composition, the end effector positioned at and actuated within the tissue to release the tissue repair composition onto the tissue.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Exemplary Surgical Stapler

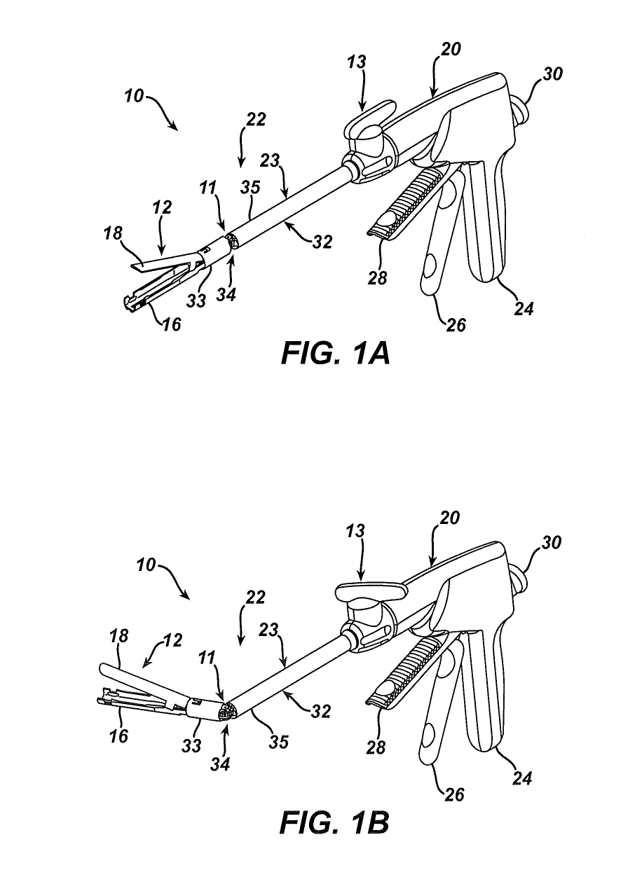

FIGS. 1-6 depict an exemplary surgical stapling and severing instrument (10) that is sized for insertion, in a nonarticulated state as depicted in FIG. 1A, through a trocar cannula passageway to a surgical site in a patient for performing a surgical procedure. Surgical stapling and severing instrument (10) includes handle portion (20) connected to implement portion (22), the latter further comprising shaft (23) distally terminating in an articulation mechanism (11) and a distally attached end effector (12). Once articulation mechanism (11) and end effector (12) are inserted through the cannula passageway of a trocar, articulation mechanism (11) may be remotely articulated, as depicted in FIG. 1B, by articulation control (13). Thereby, end effector (12) may reach behind an organ or approach tissue from a desired angle or for other reasons. It should be understood that terms such as “proximal” and “distal” are used herein with reference to a clinician gripping handle portion (20) of instrument (10). Thus, end effector (12) is distal with respect to the more proximal handle portion (20). It will be further appreciated that for convenience and clarity, spatial terms such as “vertical” and “horizontal” are used herein with respect to the drawings. However, surgical instruments are used in many orientations and positions, and these terms are not intended to be limiting and absolute.

End effector (12) of the present example includes a lower jaw (16) and a pivotable anvil (18). Handle portion (20) includes pistol grip (24) toward which closure trigger (26) is pivotally drawn by the clinician to cause clamping, or closing, of the anvil (18) toward lower jaw (16) of end effector (12). Such closing of anvil (18) is provided through an outmost closure sleeve (32), which longitudinally translates relative to handle portion (20) in response to pivoting of closure trigger (26) relative to pistol grip (24). A distal closure ring (33) of closure sleeve (32) is indirectly supported by frame (34) of implement portion (22). At articulation mechanism (11), a proximal closure tube (35) of closure sleeve (32) communicates with the distal closure ring (33). Frame (34) is flexibly attached to lower jaw (16) via articulation mechanism (11), enabling articulation in a single plane. Frame (34) also longitudinally slidingly supports a firing drive member (not shown) that extends through shaft (23) and communicates a firing motion from firing trigger (28) to firing bar (14). Firing trigger (28) is farther outboard of closure trigger (26) and is pivotally drawn by the clinician to cause the stapling and severing of clamped tissue in end effector (12), as will be described in greater detail below. Thereafter, release button (30) is depressed to release the tissue from end effector (12).

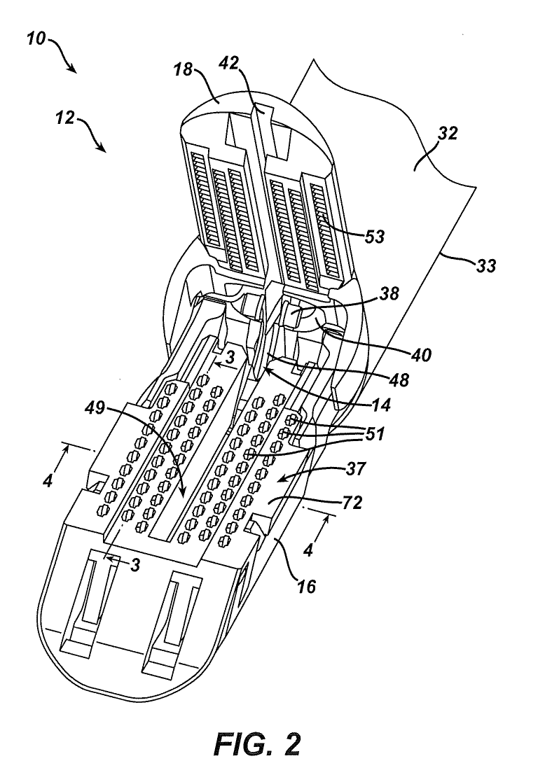

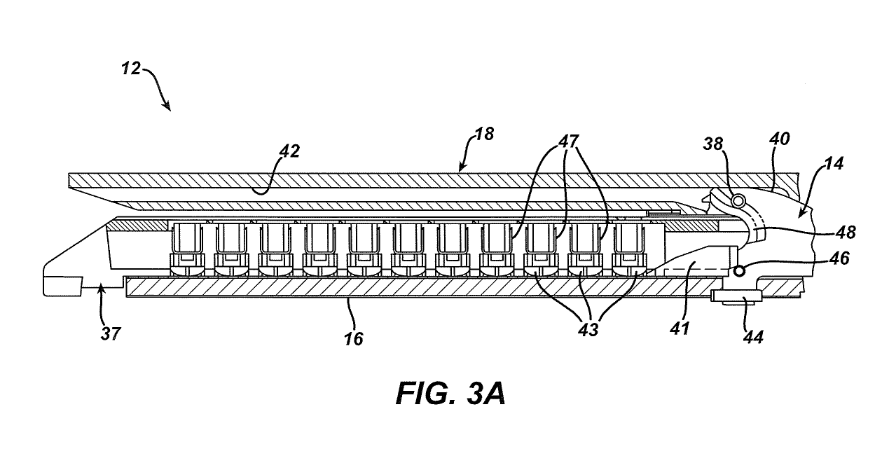

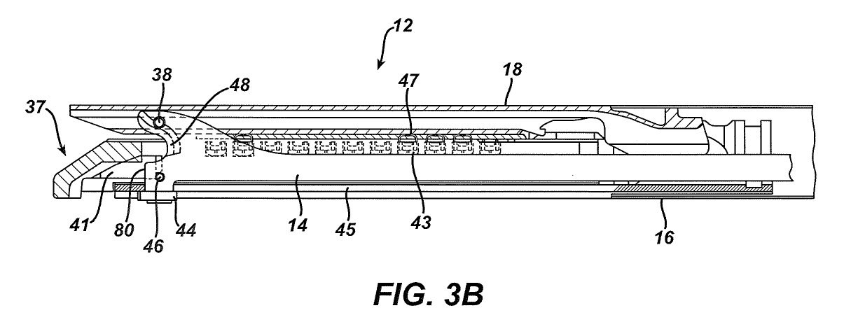

FIGS. 2-5 depict end effector (12) employing an E-beam firing bar (14) to perform a number of functions. As best seen in FIGS. 3A-3B, firing bar (14) includes a transversely oriented upper pin (38), a firing bar cap (44), a transversely oriented middle pin (46), and a distally presented cutting edge (48). Upper pin (38) is positioned and translatable within an anvil pocket (40) of anvil (18). Firing bar cap (44) slidably engages a lower surface of lower jaw (16) by having firing bar (14) extend through channel slot (45) (shown in FIG. 3B) that is formed through lower jaw (16). Middle pin (46) slidingly engages a top surface of lower jaw (16), cooperating with firing bar cap (44). Thereby, firing bar (14) affirmatively spaces end effector (12) during firing, overcoming pinching that may occur between anvil (18) and lower jaw (16) with a minimal amount of clamped tissue and overcoming staple malformation with an excessive amount of clamped tissue.

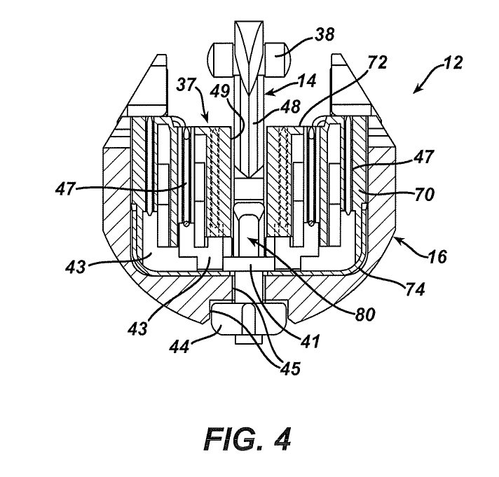

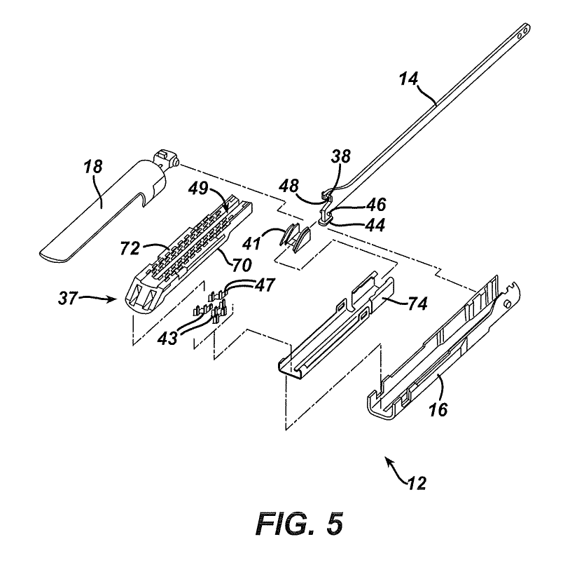

FIG. 2 shows firing bar (14) proximally positioned and anvil (18) pivoted to an open position, allowing an unspent staple cartridge (37) to be removably installed into a channel of lower jaw (16). As best seen in FIGS. 4-5, staple cartridge (37) of this example includes a cartridge body (70), which presents an upper deck (72) and is coupled with a lower cartridge tray (74). As best seen in FIG. 2, a vertical slot (49) is formed through part of staple cartridge (37). As also best seen in FIG. 2, three rows of staple apertures (51) are formed through upper deck (72) on one side of vertical slot (49), with another set of three rows of staple apertures (51) being formed through upper deck (72) on the other side of vertical slot (49). Referring back to FIGS. 3-5, a wedge sled (41) and a plurality of staple drivers (43) are captured between cartridge body (70) and tray (74), with wedge sled (41) being located proximal to staple drivers (43). Wedge sled (41) is movable longitudinally within staple cartridge (37); while staple drivers (43) are movable vertically within staple cartridge (37). Staples (47) are also positioned within cartridge body (70), above corresponding staple drivers (43). In particular, each staple (47) is driven vertically within cartridge body (70) by a staple driver (43) to drive staple (47) out through an associated staple aperture (51). As best seen in FIGS. 3A-3B and 5, wedge sled (41) presents inclined cam surfaces that urge staple drivers (43) upwardly as wedge sled (41) is driven distally through staple cartridge (37).

With end effector (12) closed as depicted in FIG. 3A, firing bar (14) is advanced in engagement with anvil (18) by having upper pin (38) enter a longitudinal anvil slot (42). A pusher block (80) is located at the distal end of firing bar (14), and is configured to engage wedge sled (41) such that wedge sled (41) is pushed distally by pusher block (80) as firing bar (14) is advanced distally through staple cartridge (37). During such firing, cutting edge (48) of firing bar (14) enters vertical slot (49) of staple cartridge (37), severing tissue clamped between staple cartridge (37) and anvil (18). As shown in FIGS. 3A-3B, middle pin (46) and pusher block (80) together actuate staple cartridge (37) by entering into slot (49) within staple cartridge (37), driving wedge sled (41) into upward camming contact with staple drivers (43) that in turn drive staples (47) out through staple apertures (51) and into forming contact with staple forming pockets (53) on the inner surface of anvil (18). FIG. 3B depicts firing bar (14) fully distally translated after completing severing and stapling tissue.

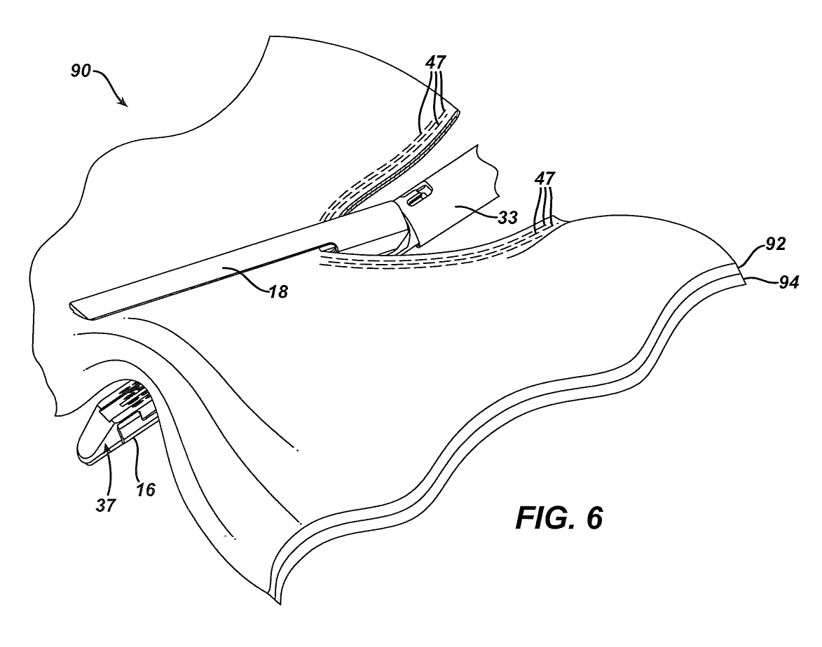

FIG. 6 shows end effector (12) having been actuated through a single stroke through tissue (90). Cutting edge (48) has cut through tissue (90), while staple drivers (43) have driven three alternating rows of staples (47) through the tissue (90) on each side of the cut line produced by cutting edge (48). Staples (47) are all oriented substantially parallel to the cut line in this example, though it should be understood that staples (47) may be positioned at any suitable orientations. In the present example, end effector (12) is withdrawn from the trocar after the first stroke is complete, spent staple cartridge (37) is replaced with a new staple cartridge, and end effector (12) is then again inserted through the trocar to reach the stapling site for further cutting and stapling. This process may be repeated until the desired amount of cuts and staples (47) have been provided. Anvil (18) may need to be closed to facilitate insertion and withdrawal through the trocar; and anvil (18) may need to be opened to facilitate replacement of staple cartridge (37).

It should be understood that cutting edge (48) may sever tissue substantially contemporaneously with staples (47) being driven through tissue during each actuation stroke. In the present example, cutting edge (48) just slightly lags behind driving of staples (47), such that a staple (47) is driven through the tissue just before cutting edge (48) passes through the same region of tissue, though it should be understood that this order may be reversed or that cutting edge (48) may be directly synchronized with adjacent staples. While FIG. 6 shows end effector (12) being actuated in two layers (92, 94) of tissue (90), it should be understood that end effector (12) may be actuated through a single layer of tissue (90) or more than two layers (92, 94) of tissue. It should also be understood that the formation and positioning of staples (47) adjacent to the cut line produced by cutting edge (48) may substantially seal the tissue at the cut line, thereby reducing or preventing bleeding and/or leaking of other bodily fluids at the cut line. Various suitable settings and procedures in which instrument (10) may be used will be apparent to those of ordinary skill in the art in view of the teachings herein.

It should be understood that instrument (10) may be configured and operable in accordance with any of the teachings of U.S. Pat. No. 4,805,823; U.S. Pat. No. 5,415,334; U.S. Pat. No. 5,465,895; U.S. Pat. No. 5,597,107; U.S. Pat. No. 5,632,432; U.S. Pat. No. 5,673,840; U.S. Pat. No. 5,704,534; U.S. Pat. No. 5,814,055; U.S. Pat. No. 6,978,921; U.S. Pat. No. 7,000,818; U.S. Pat. No. 7,143,923; U.S. Pat. No. 7,303,108; U.S. Pat. No. 7,367,485; U.S. Pat. No. 7,380,695; U.S. Pat. No. 7,380,696; U.S. Pat. No. 7,404,508; U.S. Pat. No. 7,434,715; and/or U.S. Pat. No. 7,721,930.

As noted above, the disclosures of each of those patents are incorporated by reference herein. Additional exemplary modifications that may be provided for instrument (10) will be described in greater detail below. Various suitable ways in which the below teachings may be incorporated into instrument (10) will be apparent to those of ordinary skill in the art. Similarly, various suitable ways in which the below teachings may be combined with various teachings of the patents cited herein will be apparent to those of ordinary skill in the art. It should also be understood that the below teachings are not limited to instrument (10) or devices taught in the patents cited herein. The below teachings may be readily applied to various other kinds of instruments, including instruments that would not be classified as surgical staplers. Various other suitable devices and settings in which the below teachings may be applied will be apparent to those of ordinary skill in the art in view of the teachings herein.

II. Exemplary Cartridge with Film

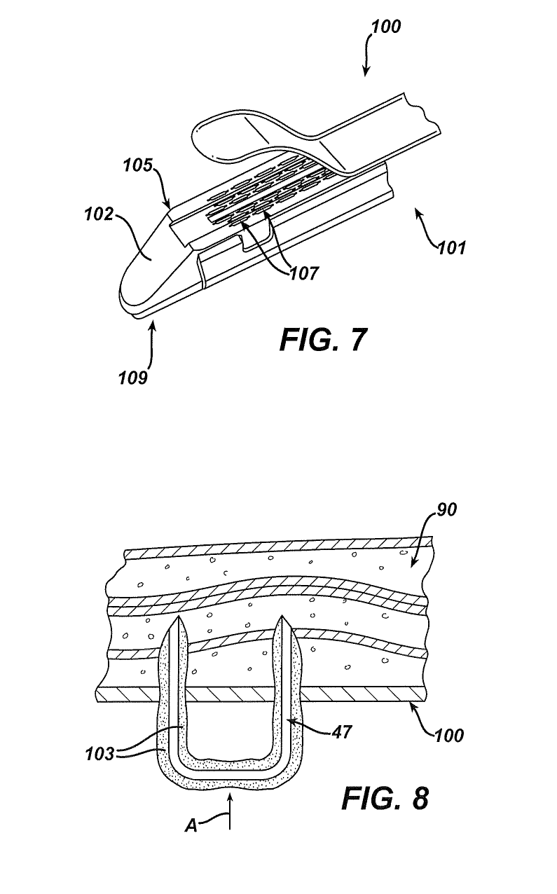

FIG. 7 shows another exemplary removable cartridge (101) that may be inserted into lower jaw (16) of end effector (12) shown in FIG. 2. Other than as set forth below, cartridge (101) of this example is similar to cartridge (37) describe above. A buttress (100) is disposed above the top surface (102) of the upper deck (105) of cartridge (101). Buttress (100) comprises a film that delivers a hemostatic agent to tissue (90), as described below. Alternatively, buttress (100) may have any other suitable properties.

Staple pockets (107) of cartridge (101) are similar to staple apertures (51) described above and are configured to receive a foam, paste, or gel material after cartridge (101) is disposed in lower jaw (16). The material contained in pockets (107) is configured to keep staples (47) in place in pockets (107) and/or to seal the medicated, biocompatible material within body (109) of cartridge (101). Pockets (107) may include various materials, such as glue, fabric, and materials that would be apparent to those of ordinary skill in the art in view of the teachings herein. A film such as buttress (100) comprising a hemostatic agent is disposed on top surface (102) to cover gel-filled pockets (107) and is heated to secure buttress (100) and the underlying medicated material into place. In some versions, only the outer edges of buttress (100) are heated to secure buttress (100) to cartridge (101). Any suitable devices may be used to provide such heating, including but not limited to an anvil (e.g., a custom thermoform die on a light press, etc.). While buttress (100) is shown as disposed over cartridge (101), buttress (100) may additionally or alternatively be disposed on an undersurface of anvil (18) that faces cartridge (101).

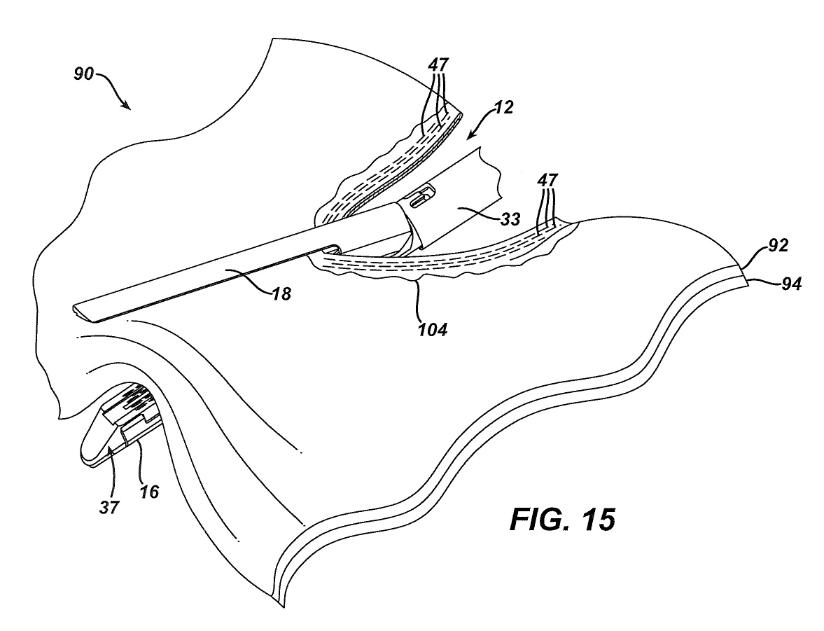

FIG. 8 shows a staple (47) being driven in a manner described above, in the direction of arrow (A), toward anvil (18) through buttress (100) and into layers of tissue (90). In some versions, staples (47) and drivers (43) may also be coated with a hemostatic agent or other adjunct material (described below), which may act as an activating agent to react with buttress (100). For example, staples (47) and drivers (43) may be coated with one of fibrin or thrombin, while buttress (100) may comprise the other of fibrin or thrombin. As coated staples (47) are driven in the direction of arrow (A) through gel-filled pockets (107) to puncture buttress (100), staples (47) release a tissue repair composition material from both staples (47) and buttress (100) onto and into tissue (90). For example, FIG. 8 shows gel (103) from gel-filled pockets (107) coated on staple (47). Additionally, when end effector (12) including cartridge (101) and buttress (100) is used, firing bar (14) is fired into tissue (90) while slicing through buttress (100) to release a tissue repair composition material from buttress (100) onto tissue (90). The tissue repair composition material may be released as the tissue repair composition (104) shown in FIG. 15 when end effector (12) including cartridge (101) staples tissue (90) with staples (47). In addition or in the alternative, material from buttress (100) may provide reinforcement to the integrity of the mechanical attachment of layers (92, 94) of tissue (90) by staples (47). Surgical staple (47) may comprise a material selected from iron, nickel titanium alloy, stainless steel, and/or titanium. Of course, any other suitable materials may be used.

The material for buttress (100) as well as the material disposed in pockets (107) and coated on staples (47) may comprise, for example, adjunct or hemostatic agents such as fibrin or thrombin that assist to coagulate blood and reduce the amount of bleeding at the surgical site. The hemostatic abilities of such adjuncts may also contribute to the use of such adjuncts as adhesives and sealants. The agents may assist to coagulate blood at a surgical site which allows tissue surrounding such blood to stick together and may prevent leaks along the stapled tissue site, for example.

Such adjuncts or reagents may further include but are not limited to medical fluid or buttress components such as platelet poor plasma (PPP), platelet rich plasma (PRP), starch, chitosan, alginate, fibrin, polysaccharide, cellulose, collagen, bovine collagen, gelatin-resorcin-formalin adhesive, oxidized cellulose, mussel-based adhesive, poly (amino acid), agarose, amylose, hyaluronan, polyhydroxybutyrate (PHB), hyaluronic acid, poly(vinyl pyrrolidone) (PVP), poly(vinyl alcohol) (PVA), polylactide (PLA), polyglycolide (PGA), polycaprolactone (PCL), and their copolymers, VICRYL® (Ethicon, Inc., Somerville, N.J.), MONOCRYL material, PANACRYL (Ethicon, Inc., Somerville, N.J.), and/or any other material suitable to be mixed with biological material and introduced to a wound or defect site, including combinations of materials. For example, buttress (100) may comprise a material selected from the following materials: epsilon-caprolactone glycolide, bovine pericardium, polylactic acid, polyglycolic acid, polyglactin, polydioxanone, polyglyconate, whey protein, cellulose gum, starch, gelatin, silk, nylon, polypropylene, braided polyester, polybutester, polyethylene, and/or polyetheretherketones. Other suitable compounds, materials, substances, etc., that may be used in a medical fluid or buttress will be apparent to those of ordinary skill in the art in view of the teachings herein.

In some versions, a medical fluid may be suspended in a biocompatible carrier to form the material of buttress (100). Suitable carriers may include, for example, a physiological buffer solution, a flowable gel solution, saline, and water. In the case of gel solutions, the tissue repair composition may be in a flowable gel form prior to delivery at the target site, or may form a gel and remain in place after delivery at the target site. Flowable gel solutions may comprise one or more gelling materials with or without added water, saline, or a physiological buffer solution. Suitable gelling materials include biological and synthetic materials. Exemplary gelling materials include proteins, polysaccharides, polynucleotides, and other materials such as alginate, cross-linked alginate, poly(N-isopropylacrylamide), poly(oxyalkylene), copolymers of poly(ethylene oxide)-poly(propylene oxide), poly(vinyl alcohol), polyacrylate, or monostearoyl glycerol co-Succinate/polyethylene glycol (MGSA/PEG) copolymers, and combinations of any of the foregoing.

Buttress (100) may comprise a fibrous pad, a foam, a matrix, a mesh, or another structure, in accordance with the teachings of, by way of example, U.S. Patent App. Pub. No. 2009/0120994, entitled “Surgical Fastening Device with Initiator Impregnation of a Matrix or Buttress to Improve Adhesive Application”, published May 14, 2009, the disclosure of which is incorporated by reference herein. The material may comprise, for example, a biocompatible material that is a buttress, a matrix having a plurality of openings therein, an open cell or closed cell foam, and/or a fabric pad. The material may include porosities that induce a wicking feature to drawing adhesive into the material and ensure the openings remain clear of the adhesive, allowing tissue growth through the openings after application to tissue.

Additionally or alternatively, buttress (100) may be comprised of an adhesive such as, but not limited to, polymerizable and/or cross-linkable materials such as a cyanoacrylate adhesive. The adhesive, for example, may be a monomeric (including prepolymeric) adhesive composition, a polymeric adhesive composition, or any other compound that can adhere to tissue. In embodiments, the monomer may be a 1,1-disubstituted ethylene monomer, e.g., an alpha-cyanoacrylate. When cross linked or polymerized, the cyanoacrylate can change from a liquid to a solid. Polymerized adhesives for example, can be formulated to be flexible to rigid and could be spongy. If desired, the adhesive can be a single part or dual part adhesive, and/or can contain additives such as alternate compounds. Polymerization of the adhesive can occur from, but is not limited to, exposure to moisture, heat, and/or adhesion initiators such as those described in U.S. Patent App. Pub. No. 2009/0120994, the disclosure of which is incorporated by reference above. Other suitable materials and compositions that may be used to form buttress (100) will be apparent to those of ordinary skill in the art in view of the teachings herein.

III. Exemplary Applicator

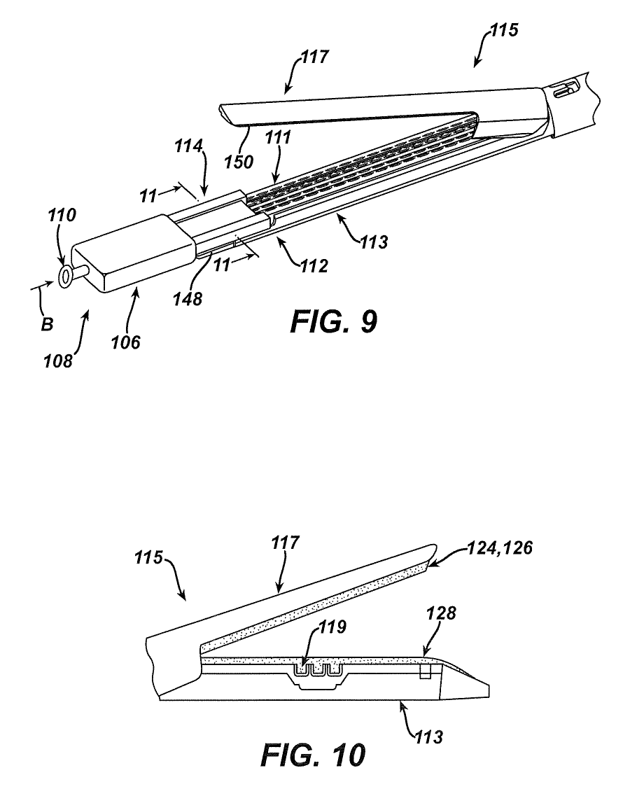

FIGS. 9-12 are associated with use of exemplary disposable applicator (106). FIG. 9 shows applicator (106) being positioned onto the upper deck (111) of a cartridge (113) in a version of end effector (115), which has components similar to end effector (12) described above. For example, the anvil (117) of end effector (115) in this example is similar to anvil (18) of end effector (12). Applicator (106) includes a first end (108) with a handle (110) to assist with urging applicator (106) onto cartridge (113). Handle (110) also assists with removing applicator (106) from cartridge (113) after material from applicator (106) is applied to anvil (117) and cartridge (113), as described below. A second end (112) of applicator (106) includes applicator portion (114) including material for deposit onto anvil (117) and cartridge (113).

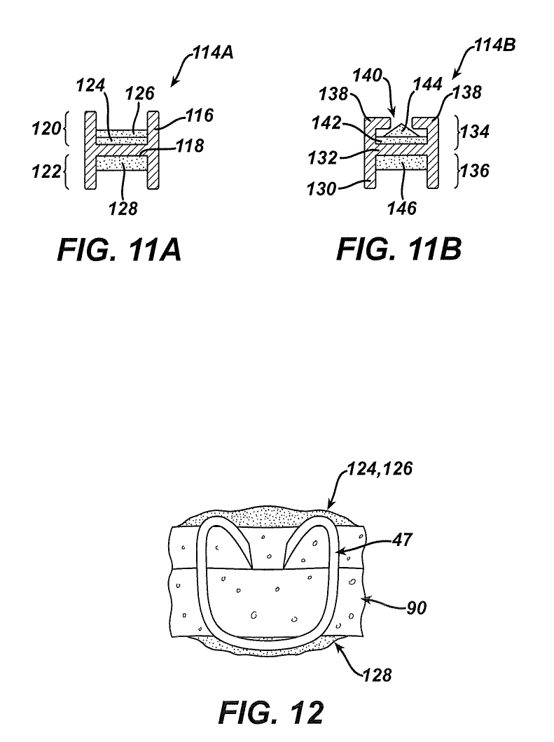

FIGS. 11A and 11B show alternative versions of cross-sections of applicator portion (114). FIG. 11A shows a version in which applicator portion (114A) presents an H-shaped cross-section including sidewalls (116) and central wall (118) disposed between sidewalls (116) to form upper portion (120) and lower portion (122). Upper portion (120) includes two layers (124, 126) of material. The first layer (124) of material may comprise a material such as fabric disposed atop wall (118). The fabric may comprise, for example, a woven Oxidized Regenerated Cellulose (ORC) that forms a buttress protecting an adjunct gel. Of course, any other suitable material (e.g., woven or nonwoven fabric, mesh, or textile, etc.) may be used. The second layer (126) of material may comprise a type of paste or gum (e.g., adhesive such as cyanoacrylate, etc.), or any other suitable material, disposed atop the first layer (124). Lower portion (122) includes a single layer (128) of material such as hemostatic adjunct gel or any other suitable material. Either side of wall (118) may include any number of layers of material.

FIG. 11B shows a version in which applicator portion (114B) presents a cross-section including sidewalls (130) and central wall (132) disposed between sidewalls (130) to form upper portion (134) and lower portion (136). Upper portion (134) includes two mutually facing arms (138), each arm (138) extending inwardly from a respective sidewall (130). Arms (138), sidewalls (130), and central wall (132) also form an inverted T-shaped channel (140) within upper portion (134). Arms (138) assist with retaining material within T-shaped channel (140), which retains two layers (142, 144) of material. The first layer (142) of material may comprise a material such as fabric disposed atop central wall (132). Similar to portion (114A), the fabric may comprise, for example, a woven ORC forming a buttress to protect an adjunct gel. The second layer (144) of material may comprise a type of gum or paste, or any other suitable material, disposed atop the first layer (142). Lower portion (136) includes a single layer (146) of material such as hemostatic adjuct gel, or any other suitable material. Either side of wall (132) may include any number of layers of material.

Referring back to FIG. 9, regardless of which applicator portion (114A, 114B) is used, applicator (106) is directed onto cartridge (113) to apply gel (128) onto deck (111) of cartridge (113). In application, sidewalls (116, 130) of applicator (106) are sized for slidable receipt in the direction of arrow (B) onto sidewalls (148) of cartridge (113). Applicator (106) locates and aligns easily on cartridge (113). When applicator portion (114A) of applicator (106) is fully received on deck (111) of cartridge (113), a user may direct anvil (117) towards cartridge (113) as described above. For example, a user may pivotally draw closure trigger (26) towards handle portion (20) to longitudinally translate closure sleeve (32) towards anvil (117) in response to the pivotal drawing motion, and closure sleeve (32) provides for closing of anvil (117). When anvil (117) presses against applicator portion (114A), undersurface (150) of anvil (117) will press against gum (126), which will act as an adhesive to attach to undersurface (150). Further, the pressure of anvil (117) against applicator portion (114A) and deck (111) will urge gel (128) into pockets (119) of deck (111). Pockets (119) filled with gel (128) are shown in FIG. 10, for example. The pressure of anvil (117) against applicator portion (114A) and deck (111) will also deposit gel (128) substantially along a top surface of deck (111).

Additionally, when anvil (117) is directed away from deck (111) of cartridge (113) by releasing closure trigger (26), which longitudinally translates closure sleeve (32) away from anvil (117), gum (126) will pull fabric (124) out of upper portion (120) of applicator portion (114A) and hold it to undersurface (150) of anvil (117). A user may then use handle (110) to pull applicator (106) away from cartridge (113) and may then dispose of applicator (106). When applicator (106) is pulled off of cartridge (113) in a direction substantially opposite to arrow (B), and where cartridge (113) is outside a patient, material (124, 126, 128) has been applied onto end effector (115), which is ready for use in a patient. FIG. 10 shows end effector (115) after applicator (106) has been used to apply material (124, 126, 128) onto end effector (12).

When end effector (115) is used in a manner similar to that described above for end effector (12), firing bar (14) will slice through gel (128) and release biocompatible gel (128) onto sliced layers (92, 94) of tissue (90). Concurrently, firing bar (14) will slice through buttress fabric (124) and adhesive gum (126) to release an adjunct gel contained in fabric (124) and adhesive from gum (126) onto sliced and severed tissue (90). Additionally, staples (47) will be driven through material (124, 126, 128) and into tissue (90) such that staples (47) capture material (124, 126, 128) and deposit it onto captured layers (92, 94) of tissue (90) as shown in FIG. 12. A new cartridge (113) may then be reloaded and a new applicator (106) may be used to apply a new layer of material (124, 126, 128) onto end effector (115) after each firing of cartridge (113) via end effector (115).

Gum (126) may comprise an adhesive such as, but not limited to, polymerizable and/or cross-linkable materials such as a cyanoacrylate adhesive. The adhesive, for example, may be a monomeric (including prepolymeric) adhesive composition, a polymeric adhesive composition, or any other compound that can adhere to tissue. In embodiments, the monomer may be a 1,1-disubstituted ethylene monomer, e.g., an alpha-cyanoacrylate. When cross linked or polymerized, the cyanoacrylate can change from a liquid to a solid. Polymerized adhesives for example, can be formulated to be flexible to rigid and could be spongy. If desired, the adhesive can be a single part or dual part adhesive, and/or can contain additives such as alternate compounds. Polymerization of the adhesive can occur from, but is not limited to, exposure to moisture, heat, and/or adhesion initiators such as those described in U.S. Patent App. Pub. No. 2009/0120994, the disclosure of which is incorporated by reference above. Other suitable materials and compositions that may be used to form gum (124) will be apparent to those of ordinary skill in the art in view of the teachings herein.

III. Exemplary Capillary Action

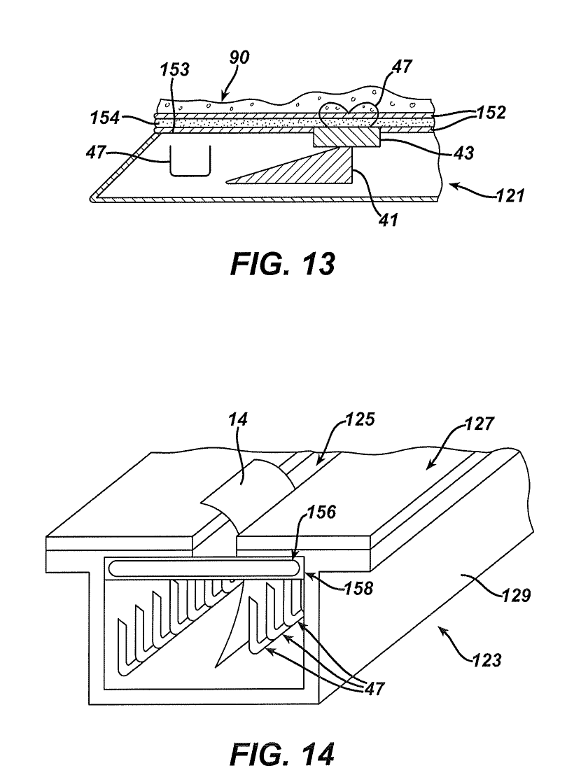

FIG. 13 shows a view of exemplary cartridge (121), which has components similar to cartridge (37) described above. Buttress (152) is disposed on upper deck (153) of cartridge (121) and may comprise a film, bladder, reservoir, or other suitable material/strucutre. Referring to FIG. 13, buttress (152) houses antibiomaterial (154) which may comprise, for example, a liquid sealant comprised of a hemostatic material as described above for delivery via capillary force to tissue (90) punctured by staple (47) of cartridge (121). For example, when staple (47) driven by wedge sled (41) and driver (43) punches through buttress (152), a gap between the legs of staple (47) and tissue (90) may provide a path for a capillary force that may pull antibiomaterial (154) along the legs of staple (47) and onto tissue (90).

IV. Exemplary Glue Filled Buttress Bag

FIG. 14 shows an exemplary cartridge (123) that has components substantially similar to cartridge (37) described above. For example, cartridge (123) includes a metal deck (127), with a longitudinal slot (125) running through a central portion of deck (127). Staples (47) are housed in a body or housing (129) of cartridge (123) under apertures (not shown) formed in deck (127) on either side of slot (125). Cartridge (123) differs from cartridge (37) as set forth below.

Cartridge (123) includes an envelope (156) disposed above staples (47) in housing (129) and below deck (127). Envelope (156) is retained in a pocket (158) formed by internal walls of housing (129) below deck (127) and above housed staples (47). Envelope (156) may comprise a glue filled buttress material comprised of an adhesive and buttress material as respectively described above for gum (126) of cartridge (113) and buttress (100) of cartridge (101). Of course, any other suitable materials and configurations may be used as apparent to one of ordinary skill in the art in view of the teachings herein.

Firing bar (14) and staples (47) may both be coated with a material, such as an adhesive or other liquid biocompatible material, to assist with application of the material from envelope (156) onto tissue (90), which firing bar (14) and staples (47) sever and staple in a manner described above for cartridge (37). Additionally, the glue in envelope (156) may be substituted with a fibrin or thrombin biologic agent. For example, firing bar (14) and staples (47) may be coated with a material such as thrombin to react with the material retained in envelope (156), which may be fibrin for example, when firing bar (14) and staples (47) puncture envelope (156). The alternative application of fibrin and thrombin is possible, such that firing bar (14) and staples (47) are coated with fibrin and envelope (156) comprises thrombin. Indeed, a wide variety of synthetic and biologic agents may be used. Such material may be applied to cartridge (123) at a manufacturing site or sold separately and applied at a later stage. Alternatively, cartridge (123) may include two compartments to contain two separate glues or any other suitable biocompatible materials.

It should be understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Versions of the devices described above may have application in conventional medical treatments and procedures conducted by a medical professional, as well as application in robotic-assisted medical treatments and procedures.

Versions of described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical instrument apparatus comprising:
(a) a handle portion;
(b) a shaft housing a firing bar;
(c) an end effector comprising an anvil, a lower jaw, and a stapling and severing assembly responsive to a longitudinal closing motion produced by the handle portion and the shaft;
(d) a removable cartridge, wherein the lower jaw is configured to receive the cartridge, the cartridge comprising:
(i) a housing,
(ii) a plurality of staples disposed in the housing,
(iii) a deck disposed over the plurality of staples, the deck defining apertures, wherein each aperture is substantially disposed over a respective staple,
(iv) at least a first biocompatible material disposed in each aperture, and
(v) at least a second biocompatible material disposed on the deck.

2. The apparatus of claim 1, wherein the first biocompatible material is the same as the second biocompatible material.

3. The apparatus of claim 2, wherein the first biocompatible material differs from the second biocompatible material.

4. The apparatus of claim 1, wherein each staple is configured to puncture the first biocompatible material and the second biocompatible material in response to the longitudinal closing motion produced by the handle portion and the shaft.

5. The apparatus of claim 1, further comprising an applicator, wherein the applicator is configured to be slidably received onto the removable cartridge to a position of alignment between the removable cartridge and the anvil.

6. The apparatus of claim 5, wherein the applicator is configured to be removed from the cartridge after use.

7. The apparatus of claim 5, wherein the applicator comprises an upper portion and a lower portion, wherein the upper portion houses a first material, and wherein the lower portion houses a second material.

8. The apparatus of claim 7, wherein an undersurface of the anvil is configured for receipt of the first material when the anvil presses against the removable cartridge responsive to the longitudinal closing motion, and wherein the first material comprises a buttress disposed below an adhesive.

9. The apparatus of claim 7, wherein the deck of the cartridge is configured for receipt of the second material when the anvil presses against the removable cartridge in response to the longitudinal closing motion, and wherein the second material comprises a biocompatible gel.

10. The apparatus of claim 1, further comprising a pocket and an envelope, wherein the pocket is defined in the cartridge, wherein the pocket is disposed below the deck and above the plurality of staples, wherein the pocket is configured to receive the envelope, and wherein the envelope comprises a biocompatible material.

11. The apparatus of claim 10, wherein the envelope comprises a buttress, and wherein the biocompatible material comprises an adhesive.

12. The apparatus of claim 11, wherein the buttress is configured to contain the adhesive.

13. The apparatus of claim 12, wherein the adhesive is comprised of a monomeric adhesive composition or a polymeric adhesive composition.

14. The apparatus of claim 10, wherein the envelope comprises a buttress, and wherein the biocompatible material comprises one of fibrin or thrombin.

15. The apparatus of claim 14, wherein each staple is coated with the other of fibrin or thrombin.

16. A surgical instrument apparatus comprising:
(a) a handle portion;
(b) a shaft housing a firing bar;
(c) an end effector comprising an anvil, a lower jaw, and a stapling and severing assembly responsive to a longitudinal closing motion produced by the handle portion and the shaft;
(d) a removable cartridge, wherein the lower jaw is configured to receive the cartridge, the cartridge comprising:
(i) a housing,
(ii) a plurality of staples disposed in the housing, and
(iii) a deck disposed over the plurality of staples, the deck defining apertures, wherein each aperture is substantially disposed over a respective staple; and
(e) an applicator configured to be slidably received onto the removable cartridge to a position of alignment between the removable cartridge and the anvil, wherein the applicator comprises an upper portion and a lower portion, wherein the upper portion houses a first material, and wherein the lower portion houses a second material.

17. The apparatus of claim 16, wherein an undersurface of the anvil is configured for receipt of the first material when the anvil presses against the removable cartridge responsive to the longitudinal closing motion, and wherein the first material comprises a buttress disposed below an adhesive.

18. The apparatus of claim 16, wherein the deck of the cartridge is configured for receipt of the second material when the anvil presses against the removable cartridge responsive to the longitudinal closing motion, and wherein the second material comprises a biocompatible gel.

19. A surgical instrument comprising:
(a) a handle portion and a shaft, the shaft housing a firing bar;
(b) a stapling and severing assembly responsive to a longitudinal closing motion produced by the handle portion and the shaft,
(c) an end effector connected to the handle portion, the end effector comprising an anvil and a lower jaw, the anvil defining an opening, the lower jaw being configured to receive the cartridge;
(d) a cartridge including a deck and a housing, wherein a plurality of staples are disposed in the housing of the cartridge, wherein the deck is disposed over the plurality of staples, the deck defining apertures, wherein each aperture is substantially disposed over a respective staple, and wherein the anvil is configured to form the staples in response to the longitudinal closing motion;
(e) an envelope comprising a biocompatible material; and
(f) a pocket defined in the cartridge, wherein the pocket is disposed below the deck and above the plurality of staples, wherein the pocket is configured to receive the envelope.

20. The apparatus of claim 19, wherein the envelope comprises a buttress, the biocompatible material comprises an adhesive, and the buttress is configured to contain the adhesive.
